(19) United States
(12) Patent Application Publication
(10) Pub. No.: US 2021/0299204 A1
(43) Pub. Date: Sep. 30, 2021

(54) Raelinh - JKL

(71) Applicant: Mary Mae Drouin, Lawrence, KS (US)

(72) Inventor: Mary Mae Drouin, Lawrence, KS (US)

(21) Appl. No.: 16/873,368

(22) Filed: Mar. 27, 2020

(51) Int. Cl. A61K 36/54

(52) U.S. Cl. CPC A61K 36/54

(57) ABSTRACT

Laurus nobilis L., or Bay Laurel, is an evergreen plant of the Lauraceae family native to the Mediterranean region and Southern Europe, widely used in herbal medicine. This is an original report on the antimicrobial bioactivity of Laurel and its regimental application effect as short-term remedy of tick-borne disease and symptoms. Utilizing brew method, loose parts of Laurel (as in leaves) were individually-measured for a 5-day regimen: Day 1—(0.5 gm.), Day 3—(1.0 gm), Day 5—(1.5 gm) with days 2, 4 in abstention; organ cleanser (as in liver) included as directed and increase in daily water intake (i.e. 160 lbs. body weight=80 oz. water) for flushing. Measure for rid of microbial endo-constituents: Jarisch-Herxheimer Reaction (JHR) observation. Day 1: Moderate/ Severe JHR; Day 2: Mild malaise; Day 3: Mild JHR; Days 4-5: no reaction. The results can be beneficial as alternative to long-term treatment with antibiotics.

### BACKGROUND

I've had food intolerance issues since I was very young; colic as a baby. Every time my Mom would take me to the doctor's for hyperactivity, etc., he would prescribe medication but my symptoms would get worse. In 2009, through a naturopathic doctor, I was tested/diagnosed with the CFIT (Carroll Food Intolerance Test) to verify my disorder and provide significant recommendations for me. I later, through research (BA Environmental Studies, KU 1999) connected salicylate intolerance/allergy. Salicylates are “naturally-occurring chemical preservatives in all foods” and my main issue is PRESERVAIVES of all kinds, especially those related to fruit/calcium (dairy) used as preservatives like ascorbic acid, etc. There is a food intolerance list and I must keep to the lower level of these as they relate to salicylate levels in foods). Everyone knows children who have food allergies have “reactions”: aggressiveness, combativeness”; their Mom's say in teset state “this is not my child”. Well, that's ME. Less than 2% of the population have this condition to the degree I, and my family, suffer.

How does this relate to my invention? It took me over 10 years to be diagnosed in the Midwest; one of the last states to admit tick-borne disease existed here. Because MOST medications contain salicylates, particularly aspirin, (produced from the flowers, [the “fruit” of the Weeping Willow]) it was difficult for my Lyme (tick-borne disease) M.D. to treat me as his antibiotic “regimen” was intolerable for me due to my disorder. HINT: Mom had shared her doc instructed her to “stay away from aspirin it bore a hole in her gut and proved her” irritable temperament/IBS (irritable bowel syndrome) she most likely suffered; love you Mom! My M.D. even tried MEPRON (yellow fever drug?) drug; 2 rounds and no Herx reaction; nothing changed. He finally said “I can't help you any longer”. That's when I went to researching to help myself; I knew it was going to be a “slippery slope” to be treated for medical issues down the line if I could not tolerate the main AMA “relief for symptoms”: drugs. I took one shot of DP (Depo-Provera) for endometrial treatment in 1987 and the result was: retinal atrophy. Turns out the preservative contained within this drug was the cause which would take years to be proven. I did go to an attorney early on, but all he had to say was “When you can prove the cause, call me back”. Really? I moved on, 2 young children, single mother, with little hope for my upcoming blindness diagnosis by KUMC, would bring me, I moved on. Many years later I heard the connection was made between my blindness and DP so I contacted another attorney and, because I had worked so hard to retrain my brain to learn to drive again, as soon as he heard I drove a car, he dumped me! I guess he wanted an easy case while others got millions; not meant to be I move on again. I now live on approximately $800 a month which was only $300 in 1992 when I was diagnosed with “permanent disability” and degenerative spine disease; later Lyme, fibromyalgia and CFS (chronic fatigue syndrome).

Enough of that. So, I researched and bingo, found what I wanted and put together a regimen to “guinea pig” myself; did it on my birthday in 2017. Before I met my Lyme specialist, I had been to many doctors around Lawrence. They could not “cure” my ailments because they had, in my opinion, “tunnel vision” on the idea of AMA drug use which my body could not tolerate. I was fired as a patient one day from LFP as they were not able to treat me conventionally so I was literally led out the door by an APRN who whispered in my ear “Go see this naturopath, he can help you”. However, I knew this was an expensive path to follow; I now had the solution but no means. I moved on once again doing what little I could for myself that research led me to.

Eventually I had suffered enough so began to follow through on a path that “had no light at the end of the tunnel (so I reasoned with limited thinking), saving tidbits and borrowing funds, it took a while but one day I walked into that naturopath's office and he took one look at me and remarked “Your liver is very ill and it has been ill for a very, very long time”. My eyes were yellow. Turns out I had a severely congested liver. The naturopath treated me with a tincture that cleaned up the congestion in my liver and, low and behold, in time the CFS went away completely. In research I found (sheet enclosed) a tidbit on “liver congestion” and how it corelates to good health in general. It all made sense to me then; it's like an aquarium tank that has not been cleaned and is full of “waste disposal”; clogged filter? Bingo: that's the liver “nowadays”. AMA test were done while my eyes were yellow; can't find that photo (hepatitis?) Was Borreliosis bacteria congesting my liver? I realized AMA liver tests are not skewed for diagnosis of liver congestion; no evidence out there. Now the tincture is claimed to treat flatulence and gastrointestinal issues. I then realized that flatulence was a symptom and so were gastrointestinal issues which come with food/drug intolerance. Then it clicked in: a new use for Chelidonium (Celandine) Homaccord was working as a “liver cleanser”. If so, then it was reasonable for me to believe from “observation” (actual treatment) it could also cleanse other organs. Why? I then, though deduction, believe endometriosis, which I suffered with for 5 yrs. not wanting to have this precious organ removed (i.e. it has many uses other than a baby incubator). However, research proved after 5 yrs. This condition could turn into endometrial cancer. I moved to drastic means: TAH. I believe endometriosis is related to “tick-borne disease”.

How is endometriosis related to “tick-borne disease”?? believe we have enough evidence about how the human body compensates/overcompensates for systematic dysfunctions. I believe the bacteria thrive on the uterine lining that is packed with nutrition, and the body overcompensates by keeping up with the production of this precious lining by producing it on the outside of the uterus where it chokes off the fallopian tubes (another dilemma for young, fertile women of childbearing age. I believe bacteria (and other microbials) revel in the most nutritional areas of our bodies: heart/blood vessels/uterine lining/liver/stomach, etc., where they can feast on these areas like “24/7 diners”. I believe these creatures' intent is NOT to kill their hosts but live off of them “in a state of euphoria”. Endometriosis has not killed a woman yert but sure is causing a lot of suffering that goes as far as to interfere with procreation.

From all this knowledge, I experimented with Laurus Nobilis and Chelidonium Homaccord and the outcome is RaeLinn-JKL.

*The following regimen was developed by me and put into action on my birthday Jun. 5, 2017. On Jun. 5, 2020 it will be 3 years since I have been free of Lyme (tick-borne) disease, fibromyalgia and CFS; 3 years seemed long enough observational proof this regimen works. I use Laurus Nobilis for many topical and internal treatment every day.

### RAELINN-JKL

### 5-DAY REGIMEN

Bay Leaf Laurus Nobilis: antibacterial, antiviral, antifungal and antimicrobial) used in conjunction with Chelidonium Homaccord (Celandine/Belladonna) antiviral tincture. Treatment for infections/co-infections including COVID-19, Lyme (tick-borne disease), etc.

Day 1 Upon waking, on empty stomach away from food/other liquids:
Bring 24 oz's. of Spring water to a boil; remove.
Add Day 1 pre-measured product to tea ball, steep 5 min./remove.
When desired temperature, drink entire 24 oz.'s of tea in your own time.
When tea is finished, wait 15 min. then SHAKE tincture bottle well before adding 10 drops to a small glass of Spring water (4-6 oz.'s) and drink; wait at least 15 min. before beginning food intake for the day.
Go about your normal day if you choose but listen to your body and rest if needed. You may soon feel as though you are coming down with a flu. Be aware to increase your daily water intake to half your body's weight in oz.'s (FOEX: 160 lbs.=80 oz.'s) to aid in flushing toxins from your system. This is the greatest thing you can do to feel better and aid healing.
   NOTE: You will most likely feel weak, light-headed, etc. You may even experience some difficulty breathing during times of exertion; These are NORMAL symptoms and side effects related to this process of “toxin flushing” due to expiration of microbial's. This is known as a “Jarisch-Herxheimer reaction. Duration and intensity correlate with level of infection; everyone is different. Symptoms may be treated with anti-inflammatories such as aspirin or ibuprofen.

Day 2 Upon waking, on empty stomach away from food/other liquids:
SHAKES tincture bottle well before adding 10 drops to small glass of Spring water (4-6 oz.'s) and drink; wait at least 15 min. before beginning food intake for the day.
You may still be experiencing a “Herx reaction”; hopefully you are also experiencing a reduction in symptoms. Continue taking the anti-inflammatory if you feel it necessary and continue to rest, or live your normal day being aware to drink half your body's weight in oz.'s of water.

Day 3 Upon waking, on empty stomach away from food/other liquids:
Bring 24 oz's. of Sprig water to a boil; remove.
Add Day 3 pre-measured product to tea ball, steep 5 min./remove
When desired temperature, drink entire 24 oz.'s of tea in your own time.
When tea is finished, wait 15 min. then SHAKE tincture bottle well before adding 10 drops to a small glass of Spring water (4-6 oz.'s) and drink; wait at least 15 min. before beginning food intake for the day.
Go about your normal day, again drinking half your body's weight daily being aware to drink plenty of water to aid flush of microbial toxins from your system.

Day 4 Upon waking, on empty stomach, away from food/other liquids:
SHAKE tincture bottle well before adding 10 drops to small glass of Spring water (4-6 oz.'s) and drink; wait at least 15 min. before beginning food intake for the day. You should be feeling your normal self again, still drinking half your body's weight oz.'s during the day.

Day 5 Upon waking, on empty stomach away from food:/other liquids:
Bring 24 oz's. of Spring water to a boil.; remove.
Add Day 5 pre-measured product to tea ball, steep 5 min./remove.
When desired temperature, drink entire 24 oz.'s of tea in your own time.
When tea is finished, wait 15 min. then SHAKE tincture bottle well before adding 10 drops to small glass of Spring water (4-6 oz.'s) and drink; wait at least 15 min. before beginning food intake for the day.
Go about your normal day. This is the last day for the tea regimen. Drinking half your body's weight in oz.'s daily would be a beneficial habit to continue!

### Specifications

Specifications for the Chelidonium Homaccord are written into the Regimen. The dry weights of the Laurus Nobilis leaf broken into bits by hand (for now) will be:
0.5 gm.—Day 1
1.0 gm.—Day 3
2.0 gm.—Day 5
   POSTYCRIPT: It is highly recommended the tincture cleansing be continued for another 25-consecutive days (a total of 30 days) by following prior directives above to avoid reinfection.
      Go forth to live the best version of your life knowing the Universe has its own intentions in its own time and trust in Ft.

### Full 90-Day Cleanse-13 3 Rounds

A full 90-day cleanse with the tincture may be undertaken. For those who chose to complete the extended 25-consecutive days of cleansing after the 5-Day Regimen, simply follow directions below to continue. If not, you must add a ROUND 3 and continue until product is consumed (i.e. You May run out of product before ending last round).

NOTE: 30-day rest period must always be taken between rounds.

ROUND 1 Upon waking on empty stomach, away from food/other liquids:
SHAKE tincture well before adding 10 drops to a small glass of Spring water (4-6 oz.'s) and drink; wait 15 min. before beginning food intake for the day. Do this for 30-consecutive days.

REST—refrain from taking the cleanse for 30-consecutive days

ROUND 2 30 consecutive days taking the cleanse each morning before food or liquids of any kind

FINIS.

1. A regimental application for remedy of tick-borne disease/symptoms, the regimen comprising: a course utilizing individually-measured loose plant parts brewed for ingest during set period allowing intermittent rest of the body from the eradication process; incorporation of an organ cleanser (as in liver along with increase of daily water intake for facilitation of lavage and flushing of congestion due to accumulation of endo-constituents.
